(19) United States
(12) Patent Application Publication
(10) Pub. No.: US 2021/0038273 A1
(43) Pub. Date: Feb. 11, 2021

(54) HELICAL IMPLANT FOR THE FIXING OF BONE ELEMENTS

(71) Applicant: Spine Arch Brevet, Oberschaeffolsheim (FR)

(72) Inventor: David François FISCHER LOKOU, Oberschaeffolsheim (FR)

(73) Assignee: Spine Arch Brevet, Oberschaeffolsheim (FR)

(21) Appl. No.: 16/321,601

(22) PCT Filed: Jul. 27, 2017
(86) PCT No.: PCT/EP2017/069078

§ 371 Date: Jan. 29, 2019

(30) Foreign Application Priority Data
Jul. 29, 2016 (FR) 1670423

(51) Int. Cl. A61B 17/86; A61B 17/17; A61B 17/70

(52) U.S. Cl. CPC A61B 17/869; A61B 17/7074; A61B 17/1757

(57) ABSTRACT

The purpose of the present invention is a new implant for a medical use enabling the fixing of at least two adjacent bone elements in a rapid, reliable and low-invasive manner. This implant is a device with three distinct parts. The present invention also describes a method for fixing at least two adjacent bone elements implementing this new implant. This method is especially suitable for the solidarization of at least two vertebrae for the realization of an arthrodesis; said implant having a curve shape that makes it particularly well adapted to the spine surgery.

The present invention provides a new implant for a surgical use allowing the fixing of at least two adjacent bone elements in a rapid, reliable and low-invasive manner. The present invention describes also a method for fixing at least two adjacent bone elements implementing this new implant. This method is particularly suitable for the solidarization of at least two vertebrae in order to make an arthrodesis; said implant is then curve-shaped what makes it particularly well adapted for spine surgery.

With regards to ageing of the population and also to the rhythm of life of the active population, the bone problems generate or are a part of the clinical picture of a lot of pathologies. In addition the traumas related to accidents, shocks and falls, could also generate problems at the bone level requiring a surgical operation. The possibility of fixing bone elements to each other in order to stabilize or to correct a given position is thus an important medical challenge. Indeed, in the field of spine surgery in particular the fact of being able to solidarize bone elements in order to stabilize given areas and/or to release compressed nerve bundles and is often the only way of relieving the patients.

In the field of the orthopaedic surgery one commonly uses plates and screws in order to stabilize adjacent bone areas or to confer to them a given position. The materials used are obviously solid and the material and the equipment impose many mechanical constraints. To allow more adaptation and to authorize more possibilities with the surgeons, different devices were implemented in order to allow a better surgical answer for each patient: surgical screws in combination with different angles, static plates, articulated plates, cages, etc.

In the particular field of spine surgery, surgeons use in general pedicle screws also called tulip screws to solidarize vertebrae. These screws of medical use are straight screws that allow arthrodesis, that is to say the bone fusion between at least two vertebrae. This technique is commonly used for fractures, scoliosis, instability and significant pain. Even if this technique is very usually practised, the installation of pedicle screws remains a difficult procedure that is reserved to the specialized surgeons and is likely to involve false ways. In the cases of false ways, one or more screws are not correctly placed, they can wound the nerve rots or have a solidity defect. To address this possibility of false way, several sophisticated systems of assistance to guidance are used by the surgeons, one will in particular quote «the navigation» including infrared detection coupled to a peroperative scanner. These navigation systems allow decreasing the risk of error of trajectory. However the error risk factor remains important because the arthrodesis practised to date require at least four pedicle screws and more often six to eight pedicle screws.

Thus the techniques used to date present several disadvantages including the complexity of the devices to implant, the transpedicle approach for the fixation screws of these devices that is relatively invasive and generator of side-effects (haemorrhage, nerve injury, etc.), the need for an follow-up implying powerful imaging techniques, all of which being risk factors, involving technicality and costs that are to be taken into account.

The presence of nerves, blood vessels and other tissues that must necessarily be preserved at the time of bone surgeries impose limited access ways and approaches to the surgeon. The errors of trajectories are all the more numerous as the access ways are difficult or exiguous. In certain cases taking into account the spatial configuration of the bone elements to solidarize the techniques known from the prior art do not offer any solution. Thus there is a real need for new technical solutions opening new possibilities of fixation of bone elements in specific configurations.

According to a first characteristic of the invention, the curved implant is one of theses solutions, and has never been suggested to date. As a matter of fact, the implant according to the invention has the advantage of allowing the installation of a solid fixing requiring only one access way. The curved shape allows as for it to work with access ways offering working angles that are not appropriate for the straight screws known from the prior art.

The present invention proposes a simple surgical implant that allows to realize the fixation of at least two adjacent bone elements, for example two adjacent vertebrae, with no risk of false way, with no particular technical difficulty and without expensive technical help other than a simple X-ray equipment. In the case of the spine surgery, contrarily to the technique implementing pedicle screws, the implants according to the present invention are not positioned in the vertebral pedicles but allow to solidarize directly the vertebral bodies between them. The device is from the start more solid than the pedicle screws commonly used in the prior art. Furthermore the vertebral bodies being large, in the range of 4×5×3 cm, the risk of an extracorporeal way is very limited compared to the vertebral pedicles that are in the range of 1×0.8 cm and are quite difficult to identify even with efficient imaging techniques.

The present invention thus proposes also a new method for the fixation of at least two adjacent bone elements, as an instance two adjacent vertebrae, with no risk of false way and with no particular technical difficulty for the surgeon. This method allows also a considerable time gain in the case of the stabilization of more than two adjacent bone elements such as several vertebral levels. Moreover this new surgical implant which once set up forms a screw, is placed directly in the bone element, with no staple nor nail, thus an arthrodesis operation as an example can be realized with a closed spine between the nerve bundles and without damaging the nerves.

In the present invention, the terms in quotation marks are used to describe the new surgical implant, the new method for the fixating implementing it and the elements constituting it. They are used in both the plural as in the singular.

By «patient» is meant a human being or an animal whose symptoms or pathology require the intervention of a surgeon in order to solidarize at least two bone elements between them. For example a patient is an individual suffering from a fracture of vertebra or presenting a scoliosis, a vertebral compressing etc. the proposed method in order to alleviate pain of said patient being the realization of an arthrodesis, that is to say the bone fusion between two vertebrae.

By «bone element» is meant anatomical bone elements requiring a surgical intervention of stabilization or reparation but also any implant that replaces for all or part an anatomical bone element with an implant made of metal or metal alloy, ceramic, polymers, or any other biocompatible material.

The invention is directed to a surgical implant for the fixing of at least two adjacent bone elements of a patient, characterized in that it consists of a rod intended to be first inserted in a drilling made in the at least two adjacent bone elements, of at least one distinct helical element intended to be second inserted on said rod, and of one sealing element.

According to a first embodiment, the present invention provides a surgical implant for the fixing of at least two adjacent bone elements of a patient, characterized in that it consists of i) a rod intended to be inserted first in a drilling made in the at least two adjacent bone elements, ii) at least one distinct helical element intended to be inserted second on said rod that serves as a guide and iii) a sealing element intended to be injected in the space released by the rod withdrawal.

According to the present invention, the rod is characterized in that it presents a curved shape.

By «rod» is meant a solid element of a cylindrical shape, filled or hollow, that constitutes the first element of the implant according to the invention.

By «curve» is meant a shape describing a plane arc or in three dimensions and not necessarily regular. The rod can be of a regular or mixed curve. When it is regular, the rod presents a regular radius of curvature, that is to say it corresponds to a circular arc regular on its whole length. We will then talk about a regular “curvature” or “arc of curvature”. When it is mixed the rod presents a straight part and at least one curved part without the rod necessarily having symmetry. In some forms of achievement, the mixed rod may present two, three or more curved parts. We will then talk about mixed “curvature” or “arc of curvature”.

According to an embodiment, the surgical implant according to the invention is characterized in that the curved shape of the rod is mixed.

According to another embodiment, the surgical implant according to the invention is characterized in that the curved shape of the rod is regular.

By «helical element» is meant a filled solid element of a helical shape, that can be flexible, aimed at being inserted along a rod in order to form a screw with said rod constituting in particular the threading. This helical element can include at least one core and according to the embodiment a thread.

Depending on various embodiments, the helical element may have a shape of the type “ wire worm”, “grooved twist thread drill”, “Archimedean screw”, “inclined threading screw” or even “solid central axis drill”. Any equivalent form known by one skilled in the art may of course be used.

By «sealing element», must be understood any material known by one skilled in the art as able to be used in bone reconstruction or known to promote bone integration and/or bone growth.

The surgical implant according to the invention is particularly adapted when the bone elements are vertebrae.

In a first version of the invention the rod presents itself as a regular cylinder that guides said at least one helical element without forcing its translation or its rotation, that is to say without forcing its helical movement.

By «helical move» is meant a move consisting of a rotational component and a translational component. In the case where the helix pitch of the helical element desired is wide enough, the helical movement actuation can be obtained by a simple push in translation. On the contrary in the case where the helix pitch of the desired helical element is narrow enough the helical movement actuation can be obtained by instigating a simple rotation to the considered element.

By «portion» is meant a part of a slightly cylindrical shape of the rod.

The shape of the rod determines the shape of the implant, thus in a particular embodiment the rod is curved and the surgical implant once implemented is also curved. In a specific embodiment the rod is curved in different planes. According to an additional characteristic the shape of the surgical implant according to the invention is curved. According to another additional characteristic the shape of the surgical implant is mixed, that is to say that it presents one or several straight part(s) and one or several curved part(s).

The curvature of the implant according to the invention is therefore not necessarily regular. According to a particular form of the invention, the implant according to the invention will comprise at least one regular curved part having preferentially a developed length being between 20 and 180 mm describing an arc typically being between 1 and 180 degrees. According to one embodiment, the implant according to the invention is characterized in that said rod consists of at least a curved part with a developed length being between 35 mm and 110 mm that will describe an arc with a radius typically being between 60 mm and 150 mm, preferentially a developed length being between 70 mm and 90 mm that will describe an arc with a radius typically being between 60 mm and 120 mm.

The materials used for the realization of the surgical implant according to the invention are biocompatible solid materials for example a metal or a metal alloy, particularly titanium or titanium based alloys and/or steel or the steel based alloys, ceramics for medical use, the polymeric materials for medical use, said materials being considered alone or in combination. According to an additional characteristic the rod and said at least one helical element are made of metal or of a metal based alloy. In a particular embodiment, the mechanical properties of the rod and of said at least one distinct helical element being different, said two elements are made in different biocompatible materials; typically in metals or metal based alloys having different properties of flexibility, elasticity, hardness, stretching and expansion. In another distinct embodiment, the rod itself is made out of different biocompatible materials in order to present different properties according to its parts, especially of flexibility. Of course in specific embodiments it can be considered different surface treatments of the rod, of the core of said helical element or of the thread of this last in order to ensure for example the biocompatibility, the cutting edge, the spring effect, the sliding or on the contrary the adherence of such or such of theses elements. In a specific embodiment said at least one helical element is made out of materials having elasticity such as for example tempered steel.

When the rod is made out of a rigid material, the surgeon must practise a drilling within the bone elements to be assembled whose curvature is identical to the one of the rod.

When the rod is made out of a solid but flexible material, the rod can potentially be adapted to any curvature of the drilling realized by the surgeon according to predefined parameters. Thus according to an additional characteristic the rod is made out of one or several biocompatible(s) and flexible(s) material(s). The rod can in particular have the shape of a nail.

Generally and whatever the shape of the implant according to the invention is it can be considered to realize only some parts of the rod in a flexible material. For example the flexible parts could be envisaged to be positioned outside the bone elements to assemble, as an example two vertebrae in order to form a kind of hinge and to preserve certain flexibility at the implant.

According to an embodiment, the surgical implant according to the invention is characterized in that the rod is realized in one or several biocompatible flexible materials.

According to another embodiment, the surgical implant is characterized in that only certain parts of the rod are flexible.

The simplicity of this surgical implant makes it particularly adaptable for the morphology of the adjacent bone element to fix between them. In fact it is easy to produce “customized” surgical implants with a slightly curved shape, having a symmetry or not compared to said curve, etc. All the forms are authorized in the present invention according to the zone to be implanted. For an implantation at the vertebral level, one will privilege for example a surgical implant with a curved shape in order to be adapted to the morphology of the vertebrae and to their articulation. The curvature is then determined according to the morphology of the bone elements to solidarize. The curvature of the implant according to the invention will not be necessarily planar and will be able to present torsions.

According to a specific embodiment, the implant according to the invention presents at least one hooking device allowing the installation and the withdrawal of said at least one helical element by the surgeon using adapted ancillaries. According to a privileged embodiment the hooking device is located on a part of the end of said at least one helical element not inserted in the bone element. The hooking device can consist of any means known by one skilled in the art, such as for example a threading, a hook, a ring, a notch, a flat, a magnet, a relief, a print, a withdrawal, a boring.

The sealing element can be natural or synthetic such as, with no limitation to, auto-grafted osseous matter, hydroxyapatite-collagen, demineralized bone matrix (DBM in the English nomenclature for Demineralized Bone Matrix), a porous synthetic osseous substitute, an osteoinductive protein or a bone morphogenic protein (BMP in the English nomenclature for Bone Morphogenic Protein), an acrylic cement, or a combination of these elements. For clarity purposes it will be thereafter referred to a sealing element consisting of acrylic cement as opposed to an osseous substitute.

According to a first particular embodiment of the invention, acrylic cement can be used. Said cement generally consists of a chemical body formed by two principal polymer components such as for example the methylmethacrylate (MMA) and the polymethylmethacrylate (PMMA) whose polymerization allows the fixing of an implant. By way of an example, it can be quoted cements PALACOS®. In the context of this first particular embodiment, it should be noted that acrylic cements have the property of being injected under a relatively homogeneous form, liquid or fluid.

According to a second particular embodiment of the invention, osseous cells or an osteoinductive protein can be used. By way of an example, it can be quoted the INDUCTOS® products. In the context of this second particular embodiment, it should be noted that the osseous substitutes are generally presented in a less homogeneous form.

According to an embodiment, the surgical implant according to the invention is characterized in that said sealing element consists of auto-grafted osseous matter, of hydroxyapatite-collagen, of demineralized bone matrix (DBM in the English nomenclature for Demineralized Bone Matrix), of a porous synthetic osseous substitute, of an osteoinductive protein or a bone morphogenic protein (BMP in the English nomenclature for

Bone Morphogenic Protein), an acrylic cement, or a combination of these elements.

The invention proposes also a method for the fixing of at least two adjacent bone elements, implementing the surgical implant made up of a rod, at least one distinct helical element and a sealing element, consisting in:

to practise a drilling having the characteristics previously defined by the surgeon in function of the bone elements to solidarize,

to insert the rod in the drilling thus practised,

to insert the at least one helical element along said rod,

to withdraw the rod from the drilling,

to inject within the space made free by the withdrawal of the rod a sealing element.

In other words, the invention provides a process for the fixing of at least two adjacent bone elements, implementing a surgical implant made up of a rod, at least one distinct helical element and a sealing element, consisting in:

to insert the rod in a drilling previously practised by the surgeon according to characteristics defined in function of the bone elements to solidarize,

to insert said at least one helical element along said rod,

to withdraw the rod from the drilling,

to inject within the space made free by the withdrawal of the rod a sealing element.

The invention provides a method for the fixing of at least two adjacent bone elements, implementing the surgical implant made up of a rod, of at least one distinct helical element and of a sealing element, consisting in:

to practise using a drilling tool a drilling presenting the characteristics defined by the surgeon according to the bone elements to solidarize, then

to insert the rod in the bore thus practised, then

to insert said at least one helical element along said rod using an installation tool allowing to instigate separately or simultaneously the different components of the helical movement to said at least one helical element, then

to withdraw the rod from the drilling in order to leave the at least one helical element in place in said drilling,

to inject within the space made free by the withdrawal of the rod a sealing element that will come to fill the space left free by the rod and to flow out through the interstices of said at least one helical element directly in contact with the bone elements to solidarize.

In other words, it is described a process for the fixing of at least two adjacent bone elements, implementing a surgical implant made up of a rod, of at least one distinct helical element and of a sealing element, consisting in:

to insert the rod in a drilling previously practised by the surgeon according to characteristics defined in function of the bone elements to solidarize,

to insert said at least one helical element along the rod using an installation tool allowing to instigate separately or simultaneously the different components of the helical movement to said at least one helical element, then

to withdraw the rod from the drilling in order to leave the at least one helical element in place in said drilling,

to inject within the space made free by the withdrawal of the rod a sealing element that will come to fill the space left free by the rod and to flow out through the interstices of said at least one helical element directly in contact with the bone elements to solidarize.

According to another embodiment comprising two distinct helical elements, the invention provides a method for the fixing of at least two adjacent bone elements, implementing the surgical implant made up of a rod, of two distinct helical elements and of a sealing element, consisting in:

to practise using a drilling tool a drilling presenting the characteristics defined by the surgeon according to the bone elements to solidarize, then

to insert the rod in the bore thus practised, then

to insert the first helical element along the rod using an installation tool allowing to instigate separately or simultaneously the different components of the helical movement to said first helical element, then

to insert the second helical element along the rod using an installation tool allowing to instigate separately or simultaneously the different components of the helical movement to said second helical element, then

to withdraw the rod from the drilling in order to leave the helical elements in place in said drilling,

to inject within the space made free by the withdrawal of the rod a sealing element that will come to fill the space left free by the rod and to flow out through the interstices of the helical elements directly in contact with the bone elements to solidarize.

In other words, it is described a process for the fixing of at least two adjacent bone elements, implementing the surgical implant made up of a rod, of two distinct helical elements and of a sealing element, consisting in:

to insert the rod in a drilling previously practised by the surgeon according to characteristics defined in function of the bone elements to solidarize,

to insert the first helical element along the rod using an installation tool allowing to instigate separately or simultaneously the different components of the helical movement to said first helical element, then

to insert the second helical element along the rod using an installation tool allowing to instigate separately or simultaneously the different components of the helical movement to said second helical element, then

to withdraw the rod from the drilling in order to leave the helical elements in place in said drilling,

to inject within the space made free by the withdrawal of the rod a sealing element that will come to fill the space left free by the rod and to flow out through the interstices of said helical elements directly in contact with the bone elements to solidarize.

One will easily understand that the rod of the implant according to the invention can be inserted like a nail by simple percussion or any other pushing on its external end, without it being necessary to print to it any rotation movement. The rod of the implant according to the invention can even be inserted this way in a drilling or a housing presenting a certain curvature, which is rigorously impossible for a traditional rigid screw. If the drilling presents a constant radius of curvature being in only one plane, that is to say without torsion, the rod of the implant according to the invention could be rigid but must have the same radius of curvature than said drilling. If the radius of curvature of the drilling is not constant or if it presents one or several torsions the rod of the surgical implant according to the invention must necessarily present certain flexibility.

One will also easily understand that the flexibility and/or the elasticity of said at least one helical element according to the invention allows its insertion by simple screwing along the rod, even curved, by printing to it a helical movement. Said at least one helical element presents in a preferred manner a sufficient rigidity to enable it to come to fit without being distorted between the rod and the internal wall of the drilling and, under the pressure exerted by this same wall, to follow the rod until its final installation.

The above-mentioned characteristics of the invention, as well as others, will more clearly appear with the reading of the following description of an example of realization, said description referring to the attached figures, among which:

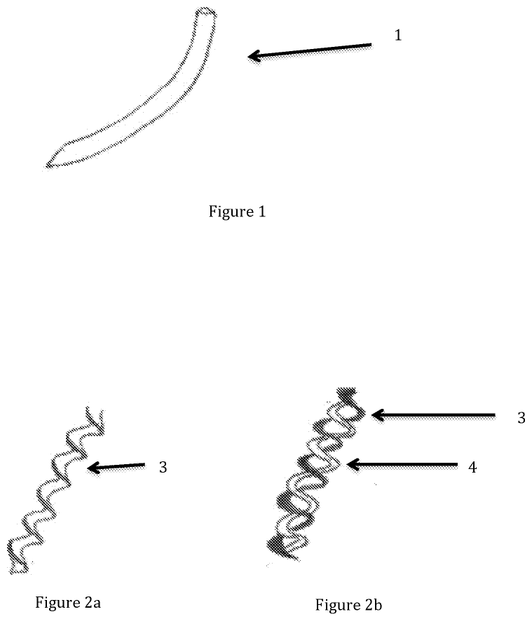

FIG. 1 represents a view of the rod (1) constituting the part of the surgical implant that is inserted first in the zone of operation;

FIG. 2 represents a view of said at least one helical element (3) with more particularly a mode of realization made up of only one helical element (FIG. 2a) or a mode of realization made up of two helical elements (FIG. 2b);

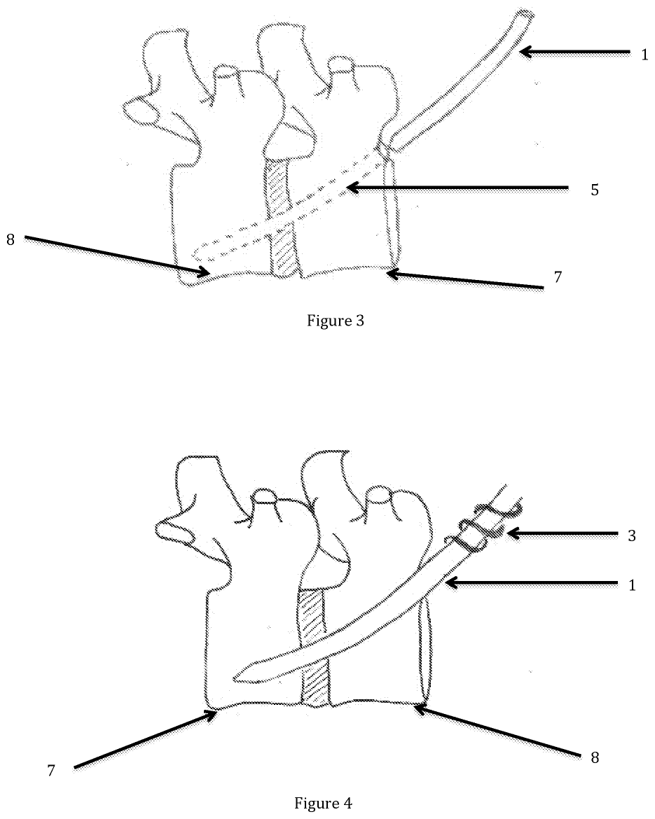

FIG. 3 represents a view of the rod (1) constituting one of the two parts of the surgical implant at the time its will be inserted in the drilling (5) practised in two adjacent vertebrae (7, 8);

FIG. 4 represents the first part of the surgical implant according to the invention, namely the rod (1) once inserted between two adjacent vertebrae (7, 8). Is also represented said at least one helical element (3) whereas this last slides along the rod (1);

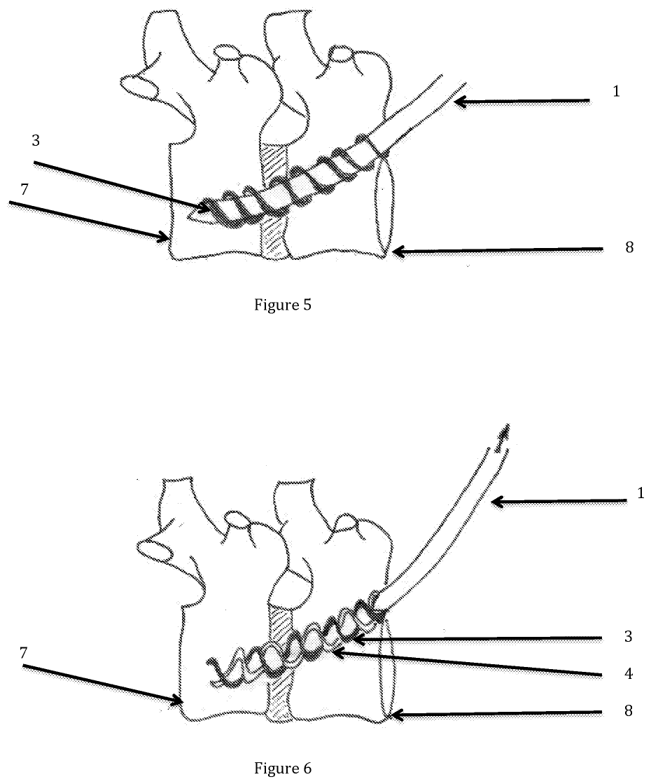

FIG. 5 represents the surgical implant once positioned between two adjacent vertebrae (7, 8), one sees the rod (1) and the first helical element (3) in its final position, thus serving as a screw within two adjacent vertebral bodies (7, 8);

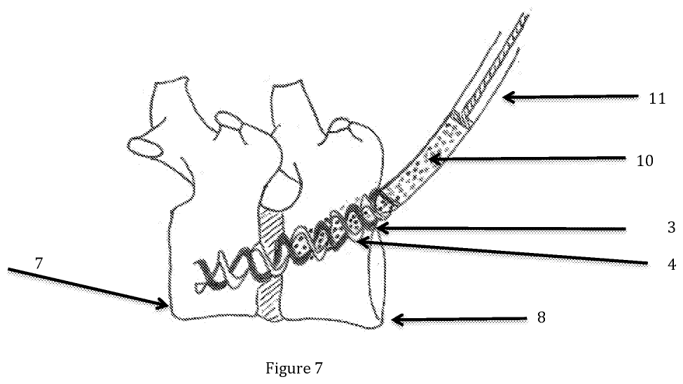

FIG. 6 illustrates the withdrawal of the rod (1) once the second helical element (4) in place;

FIG. 7 represents the injection of the sealing element (10) within the space that was initially occupied by the rod (1).

This example of installation of an implant according to the invention describes the solidarization of two adjacent vertebrae (7, 8) thanks to a surgical implant such as described above and the use of a sealing element (10). The illustrated embodiment reveals a rod (1) of curved shape and two helical elements (3, 4) that are inserted along the rod (1).

FIG. 1 illustrates the first element constituting the surgical implant according to the invention, namely the curved rod (1). In this embodiment, the proximal end is in the form of a point. This first element is inserted, generally using a drilling and installation tool, within two adjacent bone elements (7, 8), here two vertebrae.

FIG. 2 illustrates two embodiments of said at least one helical element (3). On FIG. 2a, it is represented a mode of realization with only one helical element (3) having a form of the type “solid central axis drill”. On FIG. 2b, it is represented a mode of realization with two helical elements, the first (3) being represented in black and the second (4) being represented in white. In this last mode of realization, the helical elements (3, 4) have a form of the type “wire worm”, “grooved twist thread drill”. Of course, these modes of realization are not limitative and are given here only for illustration and clarity purposes.

FIG. 3 illustrates the rod (1) at the time it will be inserted within the drilling (5) previously realized by the surgeon. The advantage of the curved shape of the rod (1) appears clearly of this figure where one can see the easiness of the implant installation.

In practise, the rod (1) can be inserted using a drilling and installation tool within the two adjacent bone elements (7, 8), here two vertebrae (toll not represented).

FIG. 4 shows the rod (1) once positioned within two adjacent vertebral bodies (7, 8) to solidarize. As it also comes out from this figure, a first helical element (3) comes to slide along the rod (1) by an helical movement that is instigated to him by the surgeon, said rod (1) serving then as a guide. While combining this movement with a light pushing, said helical element (3) will come to slide along the rod (1) into the space made up between the external walls of the rod (1) and the internal walls of cavity of the drilling (5).

FIG. 5 shows the first helical element (3) in its final position within two vertebral bodies (7, 8) along the rod (1).

The second helical element (4) is then inserted along the rod (1) in the same way as the first element (3) without coming to move this first. This stage is not represented.

FIG. 6 shows the two helical elements (3, 4) in their final position within the two adjacent vertebral bodies (7, 8) in our example. This figure illustrates also the withdrawal of the rod (1) realized by the surgeon in order for the helical elements (3, 4) to remain in place. This results in a space made free in the middle of the helical elements (3, 4), there even where the rod (1) was. The vertebral pedicles are not affected by the installation of the surgical implant thus one avoids any problem of false way and/or of nervous or vascular lesion.

FIG. 7 illustrates the surgical implant in its final position. The sealing element (10) is then injected through a syringe (11) or any equivalent device directly within the space made free by the withdrawal of the rod (1). This last will then flow out and fill the whole free space within the helical elements (3, 4). In a preferred manner, this last will be able to flow out outside this space directly in contact with the vertebrae (7, 8). In addition to the fact of solidarizing the implant within the vertebrae, this flow of the sealing element (10) will make it possible to come to fill the osseous spans of the vertebrae (7, 8) and thus to avoid the problems or complications linked to the pseudoarthrosis phenomenon.

The installation of such a surgical implant according to the invention is typically realized using two distinct tools aimed at positioning very precisely the two elements constituting said surgical implant, namely the rod (1) and said at least one distinct helical element (3, 4). A drilling tool allows practising a drilling (5) in the two bone elements (7, 8) to solidarize according to a way determined by the surgeon. Then a first tool allows the installation of the rod (1), then a second installation tool allow the insertion of the at least one helical element (3, 4) along the rod (1) while instigating a helical movement. The rod (1) previously installed is used as a guide to said at least one helical element (3, 4). According to an additional characteristic it can be envisaged a stop element in order to determine very precisely when said at least one helical element (3, 4) is in its final position. Said stop element can be located either at the distal end of the rod (1), or at its proximal end i.e. on the level of the insertion point of the rod (1) in the first bon element (8).

When the surgeon judges it applicable in function of the bone elements to assemble and of the material constituting the rod (1), it can be envisaged to use a drilling and installation tool that employs the rod (1) of the surgical implant presenting a pointed distal end as a drilling head. In this mode of realization there is no preliminary stage of drilling when the method according to the invention is applied.

In order to confer more solidity to the implant it can be envisaged elements in relief, such as pins, on the walls of said at least one helical element (3, 4) oriented in an adequate way to oppose a resistance to unscrewing of said at least one helical element (3, 4) after its installation by the surgeon.

The present invention concerns also a method for fixing at least two adjacent vertebrae (7, 8) that implements a surgical implant as described above, consisting of a rod (1), at least one distinct helical element (3, 4) and one sealing element (10), said method consisting in:

to practice by means of a drilling tool a drilling (5) displaying the characteristics defined by the surgeon according to the vertebrae (7, 8) to solidarize, then

to insert the rod (1) in the drilling (5) thus made by means of a setting tool under medical radioscopy, then to insert each of the at least one distinct helical element (3, 4) on the rod (1) by means of a setting tool allowing to give separately or simultaneously the various components of helical movement to said at least one helical element (3, 4), optionally

to block said at least one helical element (3, 4) in the desired position using a blocking device, then

to inject the sealing element (10).

In the case of spine implantation for an adult patient, the surgical implant according to the invention aimed at solidarizing two vertebrae is of a developed length of 70 mm and curve-shaped following an arc with a 90 mm radius. As a first step a drilling (5) is made with a first drilling tool. Then a setting tool is used in order to place the rod (1) from the cortical through the smooth part of the first vertebral body (8) and finally towards the second vertebral body (7) to be fixed. As a second step said at least one distinct helical element (3, 4) is inserted along the rod (1) by means of a suitable ancillary device allowing its insertion while giving it a helical movement, the rod (1) beforehand placed in the drilling serving as a guide.

Typically for an adult patient, the rod (1) has a diameter of 6.0 mm. The whole surgical implant requires therefore a drilling (5) of a diameter of about 10.0 mm in all within the two adjacent vertebrae (7, 8) to fix for an adult patient.

The present invention relates also to cases or kits containing the material necessary to the implementation of the above-described method.

According to a first embodiment, the present invention concerns cases or kits containing a surgical implant in one of the above-detailed embodiments.

According to an embodiment, the present invention concerns a case or a kit including a surgical implant as defined.

According to a second embodiment, the present invention concerns cases or kits including a surgical implant in one of the embodiments detailed above and a drilling tool.

According to a third embodiment, the present invention is directed to cases or kits including a surgical implant according to one of the embodiments detailed above, a drilling tool, a setting tool for the rod and a setting tool of said at least one helical element allowing its insertion with an helical movement; these three tools forming an ancillary devices kit.

In a preferred embodiment the case or kit includes a curve shaped surgical implant and an ancillary devices kit.

1. Surgical implant for the fixing of at least two adjacent bone elements of a patient, the surgical implant comprising:
i) a rod intended to be inserted first in a drilling (5) made in the at least two adjacent bone elements,
ii) at least one distinct helical element intended to be inserted second on said rod that serves as a guide, and
iii) a sealing element intended to be injected into space rendered free by the removal of the rod.

2. Surgical implant according to claim 1, wherein the rod presents a curved shape.

3. Surgical implant according to claim 2, wherein the curved shape of the rod is mixed.

4. Surgical implant according to claim 2, wherein the curved shape of the rod is regular.

5. Surgical implant according to claim 1, wherein the rod is carried out in one or several biocompatible flexible materials.

6. Surgical implant according to claim 1, wherein the rod portions positioned outside the bone elements to assemble are flexible.

7. Surgical implant according to claim 1, wherein said sealing element consists of self-grafted bone material, hydroxyapatite collagen, demineralized bone matrix (DBM according to the English “Demineralized Bone Matrix”), a porous synthetic bone substitute, an osteo-inductant protein or a morphogenic bone protein (BMP according to the English “Bone Morphogenic Protein”), an acrylic cement, or a combination of these elements.

8. Case or kit composed of a surgical implant according to claim 1.
